(19) United States
(12) Patent Application Publication
(10) Pub. No.: US 2018/0355437 A1
(43) Pub. Date: Dec. 13, 2018

(54) PLASMA/SERUM TARGET ENRICHMENT

(71) Applicant: GENETICS RESEARCH, LLC, D/B/A ZS GENETICS, INC., Wakefield, MA (US)

(72) Inventor: Anthony P. Shuber, Wakefield, MA (US)

(21) Appl. No.: 16/007,498

(22) Filed: Jun. 13, 2018

Related U.S. Application Data
(60) Provisional application No. 62/672,217, filed on May 16, 2018.
(60) Provisional application No. 62/656,592, filed on Apr. 12, 2018.
(60) Provisional application No. 62/526,091, filed on Jun. 28, 2017.
(60) Provisional application No. 62/519,051, filed on Jun. 13, 2017.

(51) Int. Cl. C12Q 1/6886

(52) U.S. Cl. CPC C12Q 1/6886; C12Q 2600/156

(57) ABSTRACT

The invention provides methods for capturing cfDNA directly from plasma or serum samples, without the need for certain complex sample preparation steps, using sequence-specific DNA-binding proteins such as Cas endonuclease to bind target nucleic acid sequences. The Cas proteins along with their sequence-specific guide RNAs may be introduced directly into blood, plasma, or serum, where the Cas proteins bind to ends of a target nucleic acid. The target nucleic acid is thus isolated or enriched in a sequence-specific manner. The target nucleic acid may then be subject to any suitable detection or analysis assay such as amplification or sequencing. The target nucleic acid may be enriched by digesting other, unbound nucleic acids present in the sample with exonuclease. The bound Cas proteins prevent exonuclease from digesting the target nucleic acid, thereby leaving the only the target nucleic acid substantially present in the sample.

### CROSS REFERENCE TO RELATED APPLICATIONS

This application claims the benefit of, and priority to, U.S. Provisional Application No. 62/672,217, filed May 16, 2018, U.S. Provisional Application No. 62/656,592, filed Apr. 12, 2018, U.S. Provisional Application No. 62/526,091, filed Jun. 28, 2017, and U.S. Provisional Application No. 62/519,051, filed Jun. 13, 2017, the contents of each of which are incorporated by reference.

### TECHNICAL FIELD

The invention relates to molecular genetics and oncology.

### BACKGROUND

Cell-free DNA (cfDNA) is continually released into the blood from both healthy and disease-affected tissue. Generally, circulating tumor DNA (ctDNA) is present in blood as double-stranded fragments of about 170 base pairs in length. Normally, such fragments would be removed by macrophages, but overproduction of cells in cancer leaves more of the cfDNA behind. Those fragments have a half-life of about two hours and are present in both early and late stage disease in many common tumors, including non-small cell lung cancer and breast cancer.

The presence of ctDNA in blood makes it possible for clinicians to diagnose or monitor cancer using liquid biopsy. In a liquid biopsy, a blood sample is taken from a patient and may be centrifuged to remove whole blood cells, leaving plasma or serum that includes cfDNA. Typically, the sample must be subject to a sample preparation protocol before any genetic analysis is performed. For example, laboratory technicians use a commercially-available kit to aliquot the serum through a series of steps that use proteinase solutions to digest away proteins, lysis buffers to dissociate vesicles and other lipid fragments, and cleaning and suspension buffers. In some protocols, the resultant mixture is washed through a membrane within a column under vacuum after which the cfDNA is eluted from the column with a specialty wash buffer. The entire process can require hours or more and the use of expensive kits. Some companies offer specialty instruments to aid in automating some of those steps. The kits and instruments are expensive, but theoretically provide isolated cfDNA for analysis. Unfortunately, while liquid biopsy is less invasive than needle biopsy or surgery, existing approaches require expensive and time-consuming sample preparation procedures, kits, and reagents.

### SUMMARY

The present invention provides methods for capturing cfDNA directly from plasma or serum samples without the need for significant sample preparation steps or kits. Methods of the invention use sequence-specific DNA-binding proteins, such as Cas endonuclease, to bind target nucleic acid sequences of intereset. In a preferred embodiment, Cas proteins, along with their sequence-specific guide RNAs, are introduced directly into blood, plasma, or serum. The Cas proteins may be introduced as part of sample collection, or added into blood collection tubes containing the blood, plasma, or serum. The guide RNAs mediate binding of the Cas proteins to a target nucleic acid of interest, such as tumor DNA fragment harboring a clinically significant mutation. The target nucleic acid may then be enriched relative to other materials in the sample by elution of bound Cas proteins or by elimination of non-target nucleic acid using, for example, nucleases. The target nucleic acid may be subject to any suitable detection or analysis assay, such as amplification or sequencing.

The Cas endonuclease is provided with one or more guide RNAs that bind to targets in the cfDNA that include or flank a locus of interest, such as a locus of a known cancer-associated mutation or a specific genetic allele of clinical interest. The Cas endonuclease binds to and protects target nucleic acid even when a mutation is only present as a small fraction of the sample. Thus, methods of the invention are useful when analyzing nucleic acid present in low abundance in a sample such as blood. Once captured and processed, the target may then be analyzed or sequenced to report and use the genetic information, e.g., to detect or monitor cancer.

Since Cas endonuclease binds specific targets in vivo, and a plasma/serum sample has qualities similar to cytoplasm, it Cas binds targets in plasma or serum without the need for significant sample preparation. Methods and related kits described herein are useful to detect the presence of mutation in a sample. Due to the nature by which a protein, such as a Cas complex, binds nucleic acid, methods may be used even where the target is present only in very small quantities, e.g., even as low as 0.01% frequency of mutant fragments among normal fragments in a sample (i.e., where about 50 copies of a ctDNA fragment harboring a mutation are present among about 500,000 unrelated fragments of similar size). Thus, methods of the invention may have particular applicability in discovering very rare, yet clinically important, information, such as mutations that are specific to a tumor and even may be used to detect specific mutations among cell-free DNA, such as tumor mutations among circulating tumor DNA.

In a preferred method, CRISPR/Cas systems and associated guide RNAs are introduced to a sample. When used according to methods of the invention, Cas endonuclease—whether catalytically active or inactive—will bind to a target consistently via a guide RNA and will protect that target (i.e., stay bound), thereby allowing the target to be obtained out of the sample, either via elution of the captured sequence or by elimination of non-target sequence. In certain aspects, the invention provides methods for detecting a target nucleic acid. Methods include obtaining a serum or plasma sample from a subject, introducing Cas proteins and guide RNA into the serum or plasma, and binding the Cas proteins to ends of a target nucleic acid. The Cas protein may be a Cas endonuclease or a catalytically deficient homolog thereof. The target nucleic acid may then be isolated from the sample.

In some embodiments, obtaining the sample includes obtaining a blood sample from a subject in a blood collection tube, centrifuging the tube to isolate serum or plasma from blood cells, and introducing the Cas endonuclease or catalytically deficient homolog thereof into the serum or plasma. The Cas endonuclease, or the catalytically deficient homolog thereof, may be introduced into the serum or plasma as a ribonucleoprotein (RNP) in which the endonuclease is complexed with the guide RNA. Preferably, the guide RNA includes at least two single guide RNA molecules that each complex with a Cas endonuclease and guide the Cas endonuclease to hybridize to one of the target, wherein the target includes a loci know to harbor a cancer-associated mutation.

The method may include separating the protein-bound target nucleic acid from some or all of the unbound nucleic acid. For example, the method may include binding the protein-bound target nucleic acid to a particle. The particle may include magnetic or paramagnetic material. The method may include applying a magnetic field to the sample. The particle may include an agent that binds to a protein bound to an end of the target nucleic acid. The agent may an antibody or fragment thereof. The method may include chromatography, applying the sample to a column, or gel electrophoresis. The method may include separating the protein-bound target nucleic acid from some or all of the unbound nucleic acid by size exclusion, ion exchange, or adsorption.

Each of the proteins may independently be any protein that binds a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). The protein may be a catalytically inactive form of a nuclease, such as a programmable nuclease described above. The protein may be a transcription activator-like effector (TALE). The protein may be complexed with a nucleic acid that guides the protein to an end of the nucleic acid. For example, the protein may be a Cas endonuclease in a complex with one or more guide RNAs. Preferably, the protein is a Cas endonuclease or a catalytically deficient homolog thereof.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the target nucleic acid may include identifying a mutation in the target nucleic acid. Identifying the mutation may include sequencing the nucleic acid (e.g., on a next-generation sequencing instrument), allele-specific amplification, and hybridizing a probe to the nucleic acid.

Methods of the invention may include amplifying the target nucleic acid to yield amplicons. Methods may further include sequencing the target nucleic acid to produce sequence reads and analyzing the sequence reads to provide genetic information of the subject. Methods may include analyzing the target nucleic acid to describe one or more mutations in the subject.

In some embodiments, the target nucleic acid includes a mutation specific to a tumor. The target nucleic acid may be present at no more than about 0.01% of cell-free DNA in the plasma or serum. By methods herein, the target nucleic acid is isolated or enriched from the serum or plasma.

Certain methods may further include detecting the target nucleic acid (e.g., by amplification, sequencing, probe hybridization, digital PCR, etc.). For example, detecting the target nucleic acid may include hybridizing the target nucleic acid to a probe or to a primer for a detection or amplification step, or labelling the target nucleic acid with a detectable label. Because the Cas proteins may be used to bind to the target in a sequence-specific manner, and thereby isolate or enrich for a specific mutation, detecting the presence of the nucleic acid may be useful to report the presence of the mutation in a subject from whom the sample is obtained. In multiplexed embodiments, a panel or any number of specific mutations is assayed for through use of steps of the methods and the results may provide a description or count of tumor mutations detected from ctDNA in a plasma or serum sample.

Furthermore, methods of the invention may include negative enrichment. As an example, Cas endonuclease may be provided with one or more guide RNAs that bind to targets in the cfDNA that include or flank a loci of interest, such as a locus of a known cancer-associated mutation or a specific genetic allele of clinical interest. The Cas endonuclease bind to, and protect, mutation-containing nucleic acid even when the mutation is only present as a small fraction of the sample. The bound Cas proteins prevent exonuclease from digesting the target nucleic acid and, after incubation with exonuclease, the only nucleic acid substantially present in the sample is the target nucleic acid. The target nucleic acid is thus isolated or enriched in a sequence-specific manner. The target nucleic acid may then be subject to any suitable detection or analysis assay such as amplification or sequencing.

In a preferred method, CRISPR/Cas systems using guide RNAs specific for a mutation is introduced to the sample under conditions such that nucleic acid containing the mutation is protected from exonuclease digestion while non-target nucleic acid is digested by an exonuclease. When used according to methods of the invention, Cas endonuclease—whether catalytically active or inactive—will bind to a target consistently via a guide RNA and will protect that target (i.e., stay bound) for at least long enough that a promiscuous exonuclease can be reliably used to digest unbound, non-target nucleic acid. By protection of the target with digestion of the non-target, a sample is effectively enriched for the target, and those remaining target fragments are captured, stored, isolated, preserved, detected, sequenced, or otherwise assayed with success that would be unobtainable without methods of the invention.

In certain aspects, the invention provides a method for detecting a target nucleic acid. The method includes obtaining a serum or plasma sample from a subject, introducing Cas proteins and guide RNA into the serum or plasma, and binding the Cas proteins to ends of a target nucleic acid. The Cas protein may be a Cas endonuclease or a catalytically deficient homolog thereof. Unbound nucleic acid is digested from the sample by introducing exonuclease while the Cas proteins prevent the exonuclease from digesting the target nucleic acid, thereby enriching the sample for the target nucleic acid. The target nucleic acid may then be isolated from the enriched sample by amplification, size fractionation, or hybrid capture. Methods may include inactivating the exonuclease (e.g., by heating) prior to the isolating step. Preferably, two of the Cas proteins bind to ends of the target nucleic acid and prevent the exonuclease from digesting the target nucleic acid.

### BRIEF DESCRIPTION OF THE DRAWINGS

FIG. 1 shows a table of the inputs and the dilation amounts used in the Example described herein. Dilution 11 is at 3× concentration from previous experiments because the experiment uses 3× as much input DNA volume in the reaction. The copies per ul of stock, copies per ul in 50 ul reaction, amount of previous dilution (ul), plasma, and total volume (ul) are indicated.

FIG. 2 shows a table of the dilutions used in the Example. For the percent of plasma in the final reaction, the percent of plasma in 2× sample, plasma dilution (ul), and tris dilution (ul) are shown in the table.

FIG. 3 shows a graph of the qPCR results after amplification from the post-cutting dilutions described in the Example.

FIG. 4 shows the tabulated qPCR results from the Example. Percent plasma, use of a Streck tube, amount of no Cas9 present, amount of Cas9 present, and percent cutting are indicated.

FIG. 5 shows a chart of the binding efficiency from the Example, particularly showing the relationship between percent cleavage and percent plasma. In particular, the percent cleavage is shown as a function of the amount or percent of plasma in the cutting reaction. Results are shown for samples with no tube and samples using a Streck tube.

FIG. 6 shows a chart of the detection signal in plasma from the Example, particularly showing the relationship between qPCR signal and percent plasma. In particular, the percent detection of no plasma in the sample is shown as a function of the percent plasma in the cutting reaction. Results are shown for samples with no tube and sample using a Streck tube.

### DETAILED DESCRIPTION

Methods of the invention provide for the enrichment of a target nucleic acid, in a sequence-specific manner, directly from blood, plasma, or serum. Preferred embodiments include obtaining a sample from a subject in which the sample includes plasma or serum. The plasma or serum includes cfDNA and thus also includes—among the cfDNA—ctDNA. Specific sequences of the ctDNA are isolated or enriched and analyzed or detected to detect or report genetic information from the subject, such as a presence or count of certain tumor mutations. Methods of the invention include introduce Cas endonucleases (or catalytically inactive homologs thereof such as dCas9) directly into serum or plasma. The Cas endonucleases are complexed with guide RNAs that include targeting portions specific for a target nucleic acid. In the plasma or serum, the complexes bind to ends of the target and protect it. Exonuclease may be introduced to digest unbound nucleic acid into monomers and fragments too small for further meaningful detection, sequencing or amplification.

Certain embodiments of the invention provide a method for detecting a target nucleic acid. The method includes obtaining a serum or plasma sample from a subject. Cas proteins and guide RNA are introduced into the serum or plasma. The Cas proteins bind to ends of a target nucleic acid. The target nucleic acid is thus isolated or enriched in a sequence-specific manner. The enriched target nucleic acid may then be subject to any suitable detection or analysis assay such as amplification or sequencing. The enriched target nucleic acid may be further enriched by digesting other, unbound nucleic acids present in the sample with exonuclease. The bound Cas proteins prevent exonuclease from digesting the target nucleic acid, thereby leaving the only the target nucleic acid substantially present in the sample. Certain preferred embodiments include obtaining a blood sample from a patient.

The target nucleic acid may further be isolated or detected by any suitable method in order to separate the target segment from other nucleic acids in the sample. For example, the isolation or detection method may include separating the protein-bound target nucleic acid from some or all of the unbound nucleic acid. The isolation or detection method may include binding the protein-bound target nucleic acid to a particle. The particle may include magnetic or paramagnetic material. The isolation or detection method may include applying a magnetic field to the sample. The particle may include an agent that binds to a protein bound to an end of the target nucleic acid. The agent may an antibody or fragment thereof. The isolation or detection method may include chromatography. The isolation or detection method may include applying the sample to a column. The isolation or detection method may include separating the protein-bound target nucleic acid from some or all of the unbound nucleic acid by size exclusion, ion exchange, or adsorption. The isolation or detection method may include gel electrophoresis.

One method for detection of protein-bound nucleic acids is immunomagnetic separation. Magnetic or paramagnetic particles are coated with an antibody that binds the protein bound to the segment, and a magnetic field is applied to separate particle-bound segment from other nucleic acids. Methods of immunomagnetic purification of biological materials such as cells and macromolecules are known in the art and described in, for example, U.S. Pat. No. 8,318,445; Safarik and Safarikova, Magnetic techniques for the isolation and purification of proteins and peptides, Biomagn Res Technol. 2004; 2:7, doi: 10.1186/1477-044X-2-7, the contents of each of which are incorporated herein by reference. The antibody may be a full-length antibody, a fragment of an antibody, a naturally occurring antibody, a synthetic antibody, an engineered antibody, or a fragment of the aforementioned antibodies. Alternatively or additionally, the particles may be coated with another protein-binding moiety, such as an aptamer, peptide, receptor, ligand, or the like.

Chromatographic methods may be used for detection. In such methods, the sample is applied to a column, and the segment is separated from other nucleic acids based on a difference in the properties of the segment and the other nucleic acids. Size exclusion chromatography is useful for separating molecules based on differences in size and thus is useful when the segment is larger than the residual nucleic acids left from the digestion step. Methods of size exclusion chromatography are known in the art and described in, for example, Ballou, David P.; Benore, Marilee; Ninfa, Alexander J. (2008). Fundamental laboratory approaches for biochemistry and biotechnology (2nd ed.). Hoboken, N.J.: Wiley. p. 129. ISBN 9780470087664; Striegel, A. M.; and Kirkland, J. J.; Yau, W. W.; Bly, D. D.; Modern Size Exclusion Chromatography, Practice of Gel Permeation and Gel Filtration Chromatography, 2nd ed.; Wiley: NY, 2009, the contents of each of which are incorporated herein by reference.

Ion exchange chromatography uses an ion exchange mechanism to separate analytes based on their respective charges. Thus, ion exchange chromatography can be used with the proteins bound to the segment impart a differential charge as compared to other nucleic acids. Methods of ion exchange chromatography are known in the art and described in, for example, Small, Hamish (1989). Ion chromatography. New York: Plenum Press. ISBN 0-306-43290-0; Tatjana Weiss, and Joachim Weiss (2005). Handbook of Ion Chromatography. Weinheim: Wiley-VCH. ISBN 3-527-28701-9; Gjerde, Douglas T.; Fritz, James S. (2000). Ion Chromatography. Weinheim: Wiley-VCH. ISBN 3-527-29914-9; and Jackson, Peter; Haddad, Paul R. (1990). Ion chromatography: principles and applications. Amsterdam: Elsevier. ISBN 0-444-88232-4, the contents of each of which are incorporated herein by reference.

Adsorption chromatography relies on difference in the ability of molecule to adsorb to a solid phase material. Larger nucleic acid molecules are more adsorbent on stationary phase surfaces than smaller nucleic acid molecules, so adsorption chromatography is useful when the segment is larger than the residual nucleic acids left from the digestion step. Methods of adsorption chromatography are known in the art and described in, for example, Cady, 2003, Nucleic acid purification using microfabricated silicon structures. Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; and Wolfe, 2002, Toward a microchip-based solid-phase extraction method for isolation of nucleic acids, Electrophoresis 23:727-733, each incorporated by reference.

Another method for detection is gel electrophoresis. Gel electrophoresis allows separation of molecules based on differences in their sizes and is thus useful when the segment is larger than the residual nucleic acids left from the digestion step. Methods of gel electrophoresis are known in the art and described in, for example, Tom Maniatis; E. F. Fritsch; Joseph Sambrook. “Chapter 5, protocol 1”. Molecular Cloning—A Laboratory Manual. 1 (3rd ed.). p. 5.2-5.3. ISBN 978-0879691363; and Ninfa, Alexander J.; Ballou, David P.; Benore, Marilee (2009). fundamental laboratory approaches for biochemistry and biotechnology. Hoboken, N.J.: Wiley. p. 161. ISBN 0470087668, the contents of which are incorporated herein by reference.

The proteins that bind to ends of the segment may be any proteins that bind a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference. The protein may be a catalytically inactive form of a nuclease, such as a programmable nuclease described above. The protein may be a transcription activator-like effector (TALE). The protein may be complexed with a nucleic acid that guides the protein to an end of the target nucleic acid. For example, the protein may be a Cas endonuclease in a complex with one or more guide RNAs.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the nucleic acid may include identifying a mutation in the nucleic acid. Identifying the mutation may include sequencing the nucleic acid (e.g., on a next-generation sequencing instrument), allele-specific amplification, and hybridizing a probe to the nucleic acid. Methods of DNA sequencing are known in the art and described in, for example, Peterson, 2009, Generations of sequencing technologies, Genomics 93(2):105-11; Goodwin, 2016, Coming of age: ten years of next-generation sequencing technologies, Nat Rev Genet 17(6):333-51; and Morey, 2013, A glimpse into past, present, and future DNA sequencing, Mol Genet Metab 110(1-2):3-24, each incorporated by reference. Other methods of DNA detection are known in the art and described in, for example, Xu, 2014, Label-Free DNA Sequence Detection through FRET from a Fluorescent Polymer with Pyrene Excimer to SG, ACS Macro Lett 3(9):845-848, incorporated by reference.

Methods may include detection or isolation of circulating tumor cells (CTCs) from a blood sample. Cytometric approaches use immunostaining profiles to identify CTCs. CTC methods may employ an enrichment step to optimize the probability of rare cell detection, achievable through immune-magnetic separation, centrifugation, or filtration. Cytometric CTC technology includes the CTC analysis platform sold under the trademark CELLSEARCH by Veridex LLC (Huntingdon Valley, Pa.). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

The sample may come from any source. For example, the source may be an organism, such as a human or non-human animal. The sample may be blood, serum, or plasma. The sample may be a liquid biopsy.

Certain embodiments of the invention provide a method for detecting a target nucleic acid. The method includes obtaining a serum or plasma sample from a subject. Cas proteins and guide RNA are introduced into the serum or plasma. The Cas proteins bind to ends of a target nucleic acid. The method includes digesting unbound nucleic acid with exonuclease while the Cas proteins prevent the exonuclease from digesting the target nucleic acid. Certain preferred embodiments include obtaining a blood sample from a patient.

Embodiments of the invention provide for treatment of a sample. A blood sample may be obtained from a patient. The sample may be collected in any suitable blood collection tube such as the collection tube sold under the trademark VACUTAINER by BD (Franklin Lakes, N.J.). In certain embodiments, the collection tube comprises an EDTA collection tube, and Na-EDTA collection tube or the collection tube sold under the trademark CELL-FREE DNA BCT by Streck, Inc. (La Vista, Nebr.), sometimes referred to in the art as a Streck tube. Use of a Streck tube stabilizes nucleated blood cells and prevents the release of genomic DNA into the sample. This facilitates the collection of sample that includes cell-free DNA. The sample may be centrifuged to generate a sample that includes a pellet of blood cells and a supernatant, which contains serum or plasma.

Serum is the liquid supernatant of whole blood that is collected after the blood is allowed to clot and centrifuged. Plasma is produced when the process includes an anticoagulant. To collect serum, blood is collected in tubes. After collection, the blood is allowed to clot by leaving it undisturbed at room temperature (about 15-30 minutes). The clot is removed by centrifuging, e.g., at 1,000-2,000×g for 10 minutes in a refrigerated centrifuge. The resulting supernatant is designated serum and may be transferred to a clean polypropylene tube using a Pasteur pipette. For plasma, blood is collected into commercially available anticoagulant-treated tubes e.g., EDTA-treated (lavender tops), citrate-treated (light blue tops), or heparinized tubes (green tops), followed by centrifugation to collect the supernatant.

The supernatant is preferably transferred to a fresh tube, away from the pellet, which may be discarded. Particularly where the collection tube included an anticoagulant, the transfer should give a good separation of the plasma from the whole blood cells. After transfer, the sample includes plasma or serum, which includes cfDNA.

In an exemplary embodiment, serum or plasma is transferred from a centrifuge tube to a new tub; complexes comprising Cas9 and guide RNA are added and the mixture is incubated. For example, amplification or an affinity assay may be performed to positively select out the bound, target nucleic acid. In another embodiment, exonuclease may be introduced to digest unbound, non-target DNA, and then the exonuclease may be deactivated (e.g., by heat). A positive selection may then follow (e.g., amplification or an affinity assay) to positively select out the bound, target nucleic acid.

In another exemplary embodiment, plasma or serum is removed from the centrifuge tube (the supernatant) and transferred into a new tube. Appropriate buffers/reagents are added to modify a chemical environment to promote binding of Cas endonuclease to the target nucleic acid. For example, pH can be adjusted, as may temperature, salinity, or co-factors present. The Cas complexes are added and allowed to incubate. For example, amplification or an affinity may be performed to positively select out the bound, target nucleic acid. An exonuclease may optionally be added, which ablates all free, non-target nucleic acid. The target may be positively selected such as by amplification or an affinity assay after exonuclease digestion of the non-target nucleic acid.

Methods of the invention include introducing the Cas endonuclease or catalytically deficient homolog thereof into the serum or plasma. In a preferred embodiment, the binding proteins are provided by Cas endonuclease/guide RNA complexes. Embodiments of the invention use Cas endonuclease proteins that are originally encoded by genes that are associated with clustered regularly interspaced short palindromic repeats (CRISPR) in bacterial genomes. A CRISPR-associated (Cas) endonuclease may be introduced directly into the serum or plasma in the sample.

Preferably, the Cas endonuclease is complexed with a guide RNA that targets the Cas endonuclease to a specific sequence. Any suitable Cas endonuclease or homolog thereof may be used. A Cas endonuclease (catalytically active or deactivated) may be Cas9 (e.g., spCas9), catalytically inactive Cas (dCas such as dCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes. The Cas13 proteins may be preferred where the target includes RNA. A Cas endonuclease/guide RNA complex includes a first Cas endonuclease and a first guide RNA. In the depicted embodiment, the complex comprises the Cas endonuclease or the catalytically deficient homolog thereof being introduced into the serum or plasma as a ribonucleoprotein (RNP) in which the Cas endonuclease or catalytically deficient homolog thereof is complexed with the guide RNA. The Cas endonuclease will bind to the target. The target may then be isolated or enriched, allowing for detection of the target.

In certain embodiments, the sample includes cfDNA from a subject. The sample is exposed to a first Cas endonuclease/guide RNA complex that binds to a target nucleic acid (e.g., a mutation of interest) in a sequence-specific fashion. In some embodiments, the complex binds to a mutation in a sequence-specific manner. A segment of the nucleic acid, i.e., the target nucleic acid, is protected by introducing the first Cas endonuclease/guide RNA complex and a second Cas endonuclease/guide RNA complex that also binds to the nucleic acid. In preferred embodiments of the method, the guide RNA comprises at least two guide RNA molecules that each complex with a Cas endonuclease and guide the Cas endonuclease to hybridize to one target nucleic acid, wherein the target nucleic acid includes a loci know to harbor a cancer-associated mutation.

Optionally, unprotected nucleic acid is digested. For example, one or more exonucleases may be introduced that promiscuously digest unbound, unprotected nucleic acid. Any suitable exonuclease may be used. Suitable exonucleases include, for example, Lambda exonuclease, RecJf, Exonuclease III, Exonuclease I, Exonuclease T, Exonuclease V, Exonuclease VII, T5 Exonuclease, and T7 Exonuclease, most of which are available from New England Biolabs (Ipswich, Mass.). While the exonucleases act, the target nucleic acid is protected by the bound complexes and survives the digestion step intact.

In certain embodiments, the exonuclease is deactivated. For example, exonuclease may be deactivated by following the manufacturer's instructions e.g., by heating to 90 degrees for a few minutes. After digestion, a positive selection step may be performed which may include, for example, amplification of the target nucleic acid by known methods or selection by an affinity assays.

The described steps including the digestion by the exonuclease leave a reaction product that includes principally only the mutant segment of nucleic acid, as well as any spent reagents, Cas endonuclease complexes, exonuclease, nucleotide monophosphates, and pyrophosphate as may be present.

The method optionally includes detecting the target nucleic acid (which may harbor the mutation). Any suitable technique may be used to detect the target nucleic acid. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid may indicate the presence of the mutation in the subject (i.e., a patient), and a report may be provided describing the mutation in the patient.

In an embodiment of the invention, a sample may contain a mutant fragment of DNA, a wild-type fragment of DNA, or both. A locus of interest is identified where a mutation may be present proximal to, or within, a protospacer adjacent motif (PAM). When the wild-type fragment is present, it may contain a wild-type allele at a homologous location in the fragment, also proximal to, or within, a PAM. A guide RNA is introduced to the sample that has a targeting portion complementary to the portion of the mutant fragment that includes the mutation. When a Cas endonuclease is introduced, it will form a complex with the guide RNA and bind to the mutant fragment but not to the wild-type fragment. The first Cas endonuclease/guide RNA complex includes a guide RNA with a targeting region that binds to the mutation but that does not bind to other variants at a loci of the mutation. The described methodology may be used to target a mutation that is proximal to a PAM, or it may be used to target and detect a mutation in a PAM, e.g., a loss-of-PAM or gain-of-PAM mutation.

The described methodology may be used to target a mutation that is proximal to a PAM, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM includes NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over ctDNA at such frequency that the potentially detectable mutations are abundant enough as to be representative of mutations over the tumor DNA at large. In such cases, mutation-specific enrichment may be used to detect mutations from a tumor. Moreover, methods may be used to determine a number of mutations over the representative, targetable portion of tumor DNA. Since the targetable portion of the genome is representative of the tumor DNA overall, the number of mutations may be used to infer a mutational burden for the tumor.

A feature of the method is that a specific mutation may be detected by a technique that includes detecting only the presence or absence of a fragment of DNA, and it need not be necessary to sequence DNA from a subject to describe mutations. Methods of the invention use protection at one or both ends of DNA segments. The gRNA selects for a known mutation on one end. A positive selection may be performed to positively select out the bound, target nucleic acid. If the gRNA does not find the mutation, no protection is provided and the molecule may be digested, e.g. in negative enrichment, and the remaining molecules are either counted or sequenced. Methods are well suited for the analysis of samples in which the target of interest is extremely rare, and particularly for the analysis of maternal plasma or serum (e.g., for fetal DNA) or a liquid biopsy (e.g., for ctDNA).

Methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. It is noted that the Cas9/gRNA complexes may be subsequently or previously labeled using standard procedures. The complexes may be fluorescently labeled, e.g., with distinct fluorescent labels such that detecting involves detecting both labels together (e.g., after a dilution into fluid partitions). Preferred embodiments of the detection do not require PCR amplification and therefore significantly reduces cost and sequence bias associated with PCR amplification. Sample analysis can also be performed by a number of approaches, such as next generation sequencing (NGS), etc. However, many analytical platforms may require PCR amplification prior to analysis. Therefore, preferred embodiments of analysis of the reaction products include single molecule analysis that avoids the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

Embodiments of the invention may include detecting the target nucleic acid and optionally providing a report describing a mutation as present in the patient. The mutation-containing fragments may be detected by a suitable assay, such as sequencing, gel electrophoresis, a probe-based assay. The detection of the isolated segment of the target nucleic acid may be done by sequencing. The digestion provides a reaction product that includes principally only the target nucleic acid, as well as any spent reagents, Cas endonuclease complexes, exonuclease (e.g. when negative enrichment is performed), nucleotide monophosphates, or pyrophosphate as may be present. The reaction product may be provided as an aliquot (e.g., in a micro centrifuge tube such as that sold under the trademark EPPENDORF by Eppendorf North America (Hauppauge, N.Y.) or glass cuvette). The reaction product aliquot may be disposed on a substrate. For example, the reaction product may be pipetted onto a glass slide and subsequently combed or dried to extend the fragment across the glass slide. The reaction product may optionally be amplified. Optionally, adaptors are ligated to ends of the reaction product, which adaptors may contain primer sites or sequencing adaptors. The presence of the segment in the reaction product aliquot may then be detected using an instrument.

The target nucleic acid may be detected, sequenced, or counted. Where a plurality of fragments are present or expected, the fragment may be quantified, e.g., by qPCR.

By the described methods, a sample can be assayed for a mutation using a technique that is inexpensive, quick, and reliable. Methods of the invention are conducive to high throughput embodiments, and may be performed, for example, in droplets on a microfluidic device, to rapidly assay a large number of aliquots from a sample for one or any number of genomic structural alterations.

The Cas endonuclease/guide RNA complexes can be designed to bind to mutations of clinical significance, such as a mutation specific to a tumor. When a mutation is thus detected, a report may be provided to, for example, describe the mutation in a patient or a subject.

A report may be provided in certain embodiments. The report preferably includes a description of the mutation in the subject (e.g., a patient). The method for detecting rare nucleic acid may be used in conjunction with a method of describing mutations (e.g., as described herein). Either or both detection processes may be performed over any number of loci in a patient's genome or preferably in a patient's tumor DNA. As such, the report may include a description of a plurality of structural alterations, mutations, or both in the patient's genome or tumor DNA. As such, the report may give a description of a mutational landscape of a tumor.

Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor.

Methods of the invention thus may be used to detect and report clinically actionable information about a patient or a tumor in a patient. For example, the method may be used to provide a report describing the presence of the genomic alteration in a genome of a subject. Additionally, protecting a segment of DNA, and optionally digesting unprotected DNA, provides a method for isolation or enrichment of DNA fragments, i.e., the protected segment. It may be found that the described enrichment techniques are well-suited to the isolation/enrichment of arbitrarily long DNA fragments, e.g., thousands to tens of thousands of bases in length or longer.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genomic regions that would be used to determine TMB for immuno-oncology associated therapeutic treatments. In such applications, the negative enrichment technology is utilized to enrich large regions (>50 kb) within the genome of interest.

Certain embodiments of the invention may provide a kit. The kit preferably includes reagents and materials useful for performing methods of the invention. For example, the kit may include one or more guide RNA that, taken in pairs, are designed to flank cancer-associated mutations. The kit may include one or more guide RNAs that are mutation specific and only hybridize to target that includes a mutation. The kit may include a Cas endonuclease or a nucleic acid encoding a Cas endonuclease such as a plasmid. The kit may optionally include exonuclease. The kit may include reagents for adjusting conditions in plasma or serum, conditions such as pH, salinity, co-factors, etc., to promote binding or activity of Cas endonuclease (including to promote binding of catalytically inactive Cas endonuclease, which may be included as the Cas endonuclease). The kit may further include instructional materials for performing methods of the invention, and components of the kit may be packaged in a box suitable for shipping or storage. Preferably, the kit contains one or more blood collection tubes.

The invention provides methods for capturing cfDNA directly from plasma or serum samples, without the need for certain complex sample preparation steps, using sequence-specific DNA-binding proteins such as Cas endonuclease to bind target nucleic acid sequences. The Cas proteins along with their sequence-specific guide RNAs may be introduced directly into blood, plasma, or serum, where they bind to ends of a target nucleic acid. The target nucleic acid may then be subject to any suitable detection or analysis assay such as amplification or sequencing.

In another embodiment, other, unbound nucleic acids present in the sample may be digested by exonuclease while the bound Cas proteins prevent exonuclease from digesting the target nucleic acid. After the exonuclease digestion, the only nucleic acid substantially present in the sample is the target nucleic acid. The target nucleic acid is thus isolated or enriched in a sequence-specific manner. The target nucleic acid may then be subject to any suitable detection or analysis assay such as amplification or sequencing.

### EXAMPLE

The cutting efficiency of amplicons by Cas9 in plasma is shown by experiment. Results from the experiment indicated that Cas proteins bind to expected cognate targets under guide RNA guidance in plasma or serum. In particular, Cas9 was tested for cutting activity in plasma in an experimental protocol.

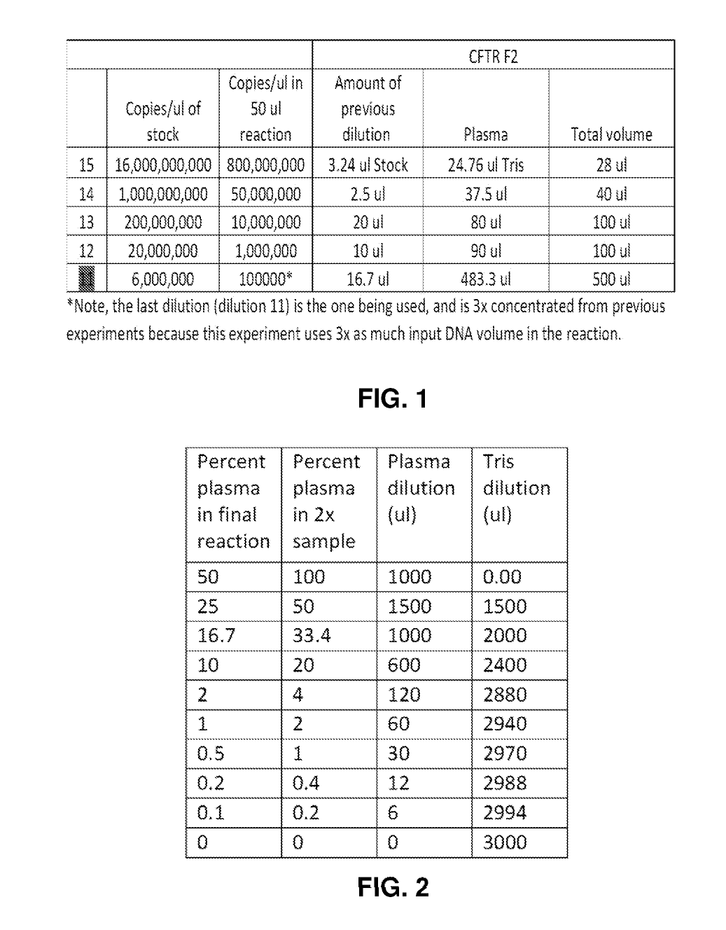

Plasma samples were placed in Streck tubes and in standard tubes. The experiments used an 800 bp amplicon from the cystic fibrosis transmembrane receptor gene. Dilutions were made of CFTR F2 800 bp into plasma with 5 million copies per reaction total (FIG. 1). The percent plasma in reaction after dilution was 50%, 25%, 16.7%, 10%, 2%, 1%, 0.5%, 0.2%, 0.1%, and 0% (FIG. 2).

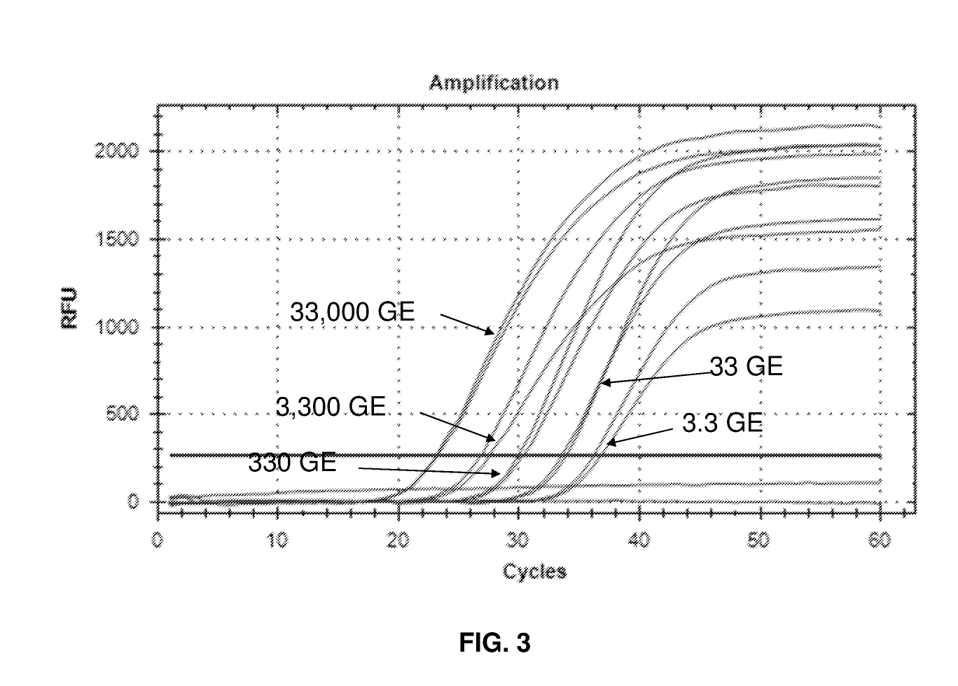

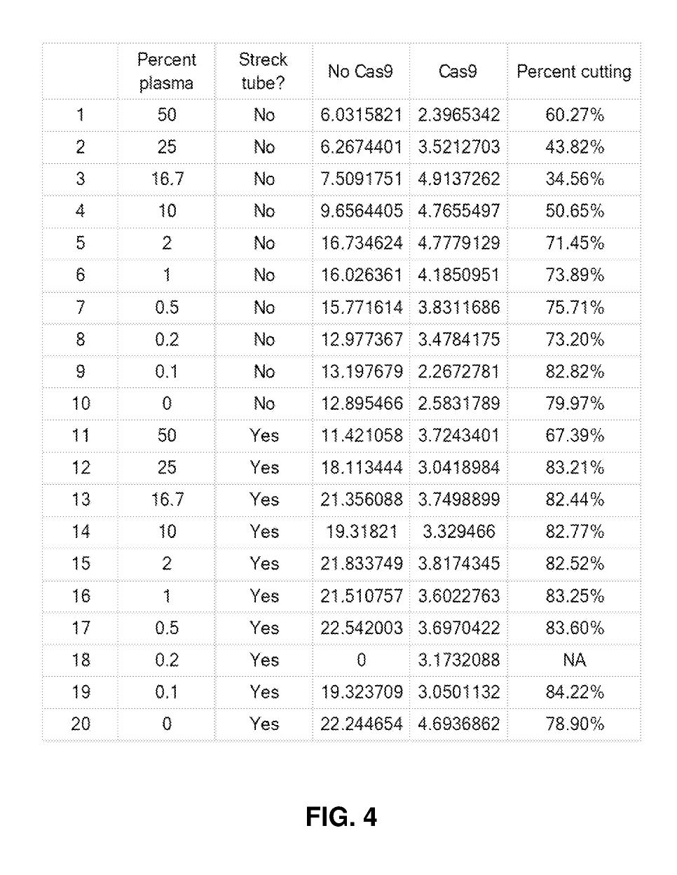

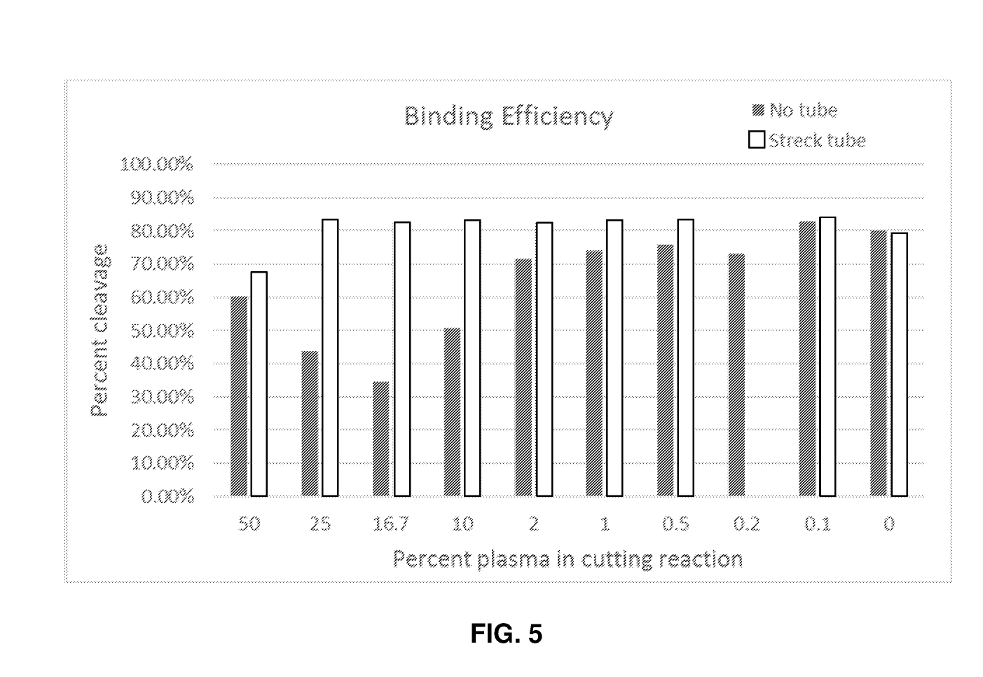

Cas9 with guide RNA was added and allowed to cut. qPCR was then used to probe across the cut site. For qPCR, samples were diluted 1/100, and then 5 ul were used per 20 ul reaction. The qPCR results were analyzed from amplifying, post-cutting, from dilutions (FIGS. 3 and 4). The qPCR results indicated cleavage as a function of plasma amount (FIG. 5). For example, every replicate in a Streck tube demonstrated greater than 60% cutting efficiency by Cas9 in the CFTR amplicon. Cas9 exhibited detectable cutting, even in standard, non-Streck tubes.

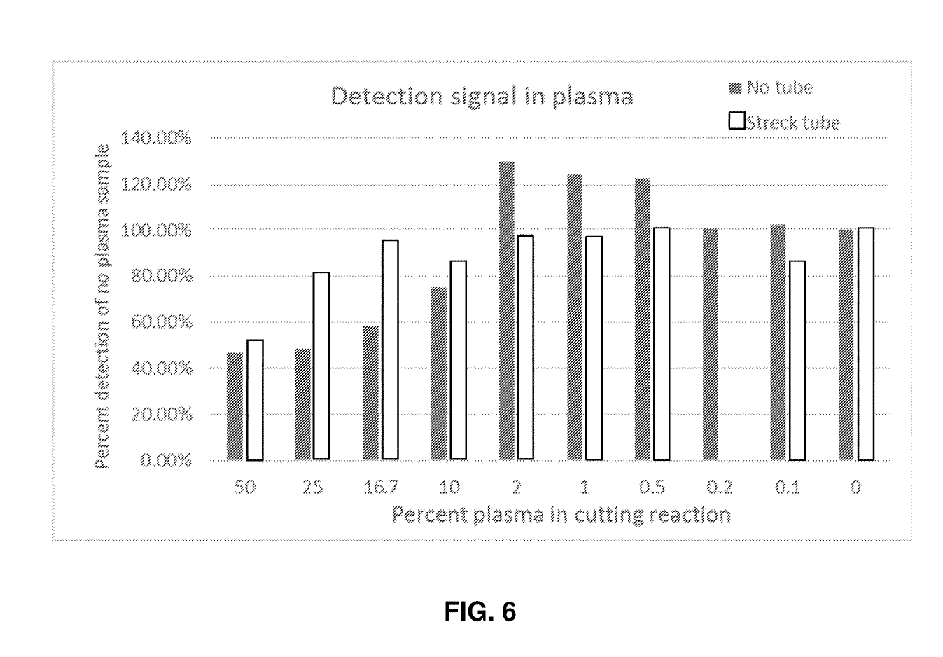

The results also indicated a relationship between the qPCR signal and percent plasma (FIG. 6). For example, the data show Cas9 exhibits detectable cutting in Na-EDTA plasma. For the reactions performed in straight plasma, cutting efficiency in 2% plasma or lower resembled no plasma cutting efficiency (82.82% for in plasma compared to 79.97% in no plasma). For the reactions performed in plasma incubated in a Streck tube, the cutting efficiency in 25% plasma or lower resembled no-plasma cutting efficiency (83.14% compared to 78.90%). Further, there was 60-67% cutting for the 50% plasma samples. In 50% plasma, CRISPR/Cas9 complexes retained 75% activity. Results of the data show that Cas endonuclease and homologs thereof bind to target DNA under guidance of guide RNA in plasma.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for enriching a sample, the method comprising:
obtaining a serum or plasma sample comprising a target nucleic acid; and
introducing a protein into the serum or plasma sample that binds the target nucleic acid in a sequence-specific manner.

2. The method of claim 1, wherein the protein comprises Cas endonuclease or a catalytically inactive homolog thereof.

3. The method of claim 2, wherein binding the protein comprises introducing Cas proteins and guide RNA into the serum or plasma and binding the Cas proteins to ends of the target nucleic acid.

4. The method of claim 2, wherein the obtaining step comprises obtaining a blood sample from a subject in a blood collection tube, centrifuging the tube to isolate serum or plasma from blood cells, and introducing the Cas endonuclease or catalytically inactive homolog thereof into the serum or plasma.

5. The method of claim 4, wherein the Cas endonuclease or the catalytically inactive homolog thereof is introduced into the serum or plasma as a ribonucleoprotein (RNP) in which the Cas endonuclease or catalytically inactive homolog thereof is complexed with the guide RNA.

6. The method of claim 3, wherein the guide RNA comprises at least two guide RNA molecules that each complex with a Cas endonuclease and guide the Cas endonuclease to hybridize to one of the target nucleic acid, wherein the target nucleic acid includes a loci known to harbor a cancer-associated mutation.

7. The method of claim 1, further comprising isolating the target nucleic acid from the enriched sample.

8. The method of claim 1, further comprising amplifying the target nucleic acid to yield amplicons.

9. The method of claim 8, further comprising sequencing the target nucleic acid to produce sequence reads and analyzing the sequence reads to provide genetic information of a subject.

10. The method of claim 1, further comprising analyzing the target nucleic acid to describe one or more mutations in a subject.

11. The method of claim 10, wherein the target nucleic acid includes a mutation specific to a tumor.

12. The method of claim 11, wherein the target nucleic acid is present at no more than about 0.01% of cell-free DNA in the plasma or serum.

13. The method of claim 1, further comprising detecting the target nucleic acid.

14. The method of claim 13, wherein the detection step comprises hybridizing the target nucleic acid to a probe or to a primer for a detection or amplification step, or labelling the target nucleic acid with a detectable label.

15. The method of claim 13, wherein the detection step comprises connecting the protein-bound target nucleic acid to a particle or column and removing other components of the sample.

16. The method of claim 15, wherein the particle comprises an agent that binds to at least one protein to form a particle-bound segment.

17. The method of claim 15, wherein the particle comprises magnetic or paramagnetic material and the detection step further comprises applying a magnetic field to separate the particle-bound segment from the other components.

18. The method of claim 13, wherein the detection step comprises applying the sample to a column.

19. The method of claim 18, wherein the protein-bound target nucleic acid is separated from unbound nucleic acid in the sample by size exclusion, ion exchange, or adsorption.

20. The method of claim 13, wherein the detection step comprises gel electrophoresis.

21. The method of claim 1, further comprising digesting unbound nucleic acid with exonuclease.

22. The method of claim 21, wherein Cas protein and guide RNA are introduced into the serum or plasma to bind to ends of the target nucleic acid and prevent the exonuclease from digesting the target nucleic acid.

23. The method of claim 21, further comprising inactivating the exonuclease prior to isolating the target nucleic acid from the enriched sample by amplification, size fractionation, or hybrid capture.

24. The method of claim 22, wherein the Cas protein comprises Cas endonuclease or a catalytically inactive homolog thereof.

25. The method of claim 24, wherein the obtaining step comprises obtaining a blood sample from a subject in a blood collection tube, centrifuging the tube to isolate serum or plasma from blood cells, and introducing the Cas endonuclease or catalytically inactive homolog thereof into the serum or plasma.

26. The method of claim 25, wherein the Cas endonuclease or the catalytically inactive homolog thereof is introduced into the serum or plasma as a ribonucleoprotein (RNP) in which the Cas endonuclease or catalytically inactive homolog thereof is complexed with the guide RNA.

27. The method of claim 22, wherein the guide RNA comprises at least two guide RNA molecules that each complex with a Cas endonuclease and guide the Cas endonuclease to hybridize to one target nucleic acid, wherein the target nucleic acid includes a loci known to harbor a cancer-associated mutation.
